(19) United States
(12) Patent Application Publication
(10) Pub. No.: US 2024/0317898 A1
(43) Pub. Date: Sep. 26, 2024

(54) Method for Preparing Type III Resistant Starch in Extrusion-Debranching Manner

(71) Applicants: Jiangnan University, Wuxi (CN); Chengtian (Taizhou) Biotechnology Co., Ltd., Taizhou City (CN)

(72) Inventors: Aiquan JIAO, Wuxi (CN); Qing Liu, Wuxi (CN); Zhengyu Jin, Wuxi (CN)

(21) Appl. No.: 18/678,247

(22) Filed: May 30, 2024

Related U.S. Application Data
(63) Continuation of application No. PCT/CN2023/093840, filed on May 12, 2023.

(30) Foreign Application Priority Data
Oct. 21, 2022 (CN) 2022112942094

(51) Int. Cl. C08B 30/12; C12P 19/16

(52) U.S. Cl. CPC C08B 30/12; C12P 19/16; C12Y 302/01041

(57) ABSTRACT

The present disclosure provides a method for preparing type III resistant starch in an “extrusion-debranching” manner and belongs to the technical field of functional food. The method for preparing type III resistant starch in an “extrusion-debranching” manner comprises the following steps: adding a starch raw material into a twin screw extruder for primary extrusion, collecting extruded, gelatinized and degraded starch, and then drying and crushing same; and fully mixing the crushed gelatinized starch with pullulanase, adding the mixture into the twin screw extruder for secondary extrusion, collecting the extruded debranched starch, then recrystallizing same, centrifuging and water washing same, and collecting the precipitate and drying same to obtain type III resistant starch. The method rapidly and efficiently debranches starch under high substrate starch concentration, increases the content of the type III resistant starch, and solves the problem of low yield efficiency of the type III resistant starch.

### TECHNICAL FIELD

The present disclosure belongs to the technical field of functional food and particularly relates to a method for preparing type III resistant starch in an “extrusion-debranching” manner.

### BACKGROUND

Depending on the rate of digestion, starch can be divided into rapidly digestible starch (RDS), slowly digestible starch (SDS) and resistant starch (RS). The consumption of the rapidly digestible starch rapidly rises the postprandial blood glucose level, whereas the slowly digestible starch is slow to digest and the blood glucose level is relatively low after the consumption. The resistant starch cannot be digested in the small intestine, but is fermented and degraded in the colon by the intestinal flora to produce short chain fatty acids and gases. The resistant starch plays a key role in a variety of effects beneficial to health, including reducing blood sugar and cholesterol, inhibiting body fat accumulation, increasing mineral absorption, reducing digestive tract cancer, and preventing gallstone formation. With the increasing threat of diabetes to human health, the development of the resistant starch has important significance to patients with obesity and diabetes. However, currently the industrialization level of the resistant starch is low and there are few resistant starch products available commercially.

The resistant starch is generally classified into 5 types: RS₁, RS₂, RS₃, RS₄ and RS₅. RS₃ is of particular interest for its thermal stability during curing and for safety when consumed. RS₃ refers primarily to recrystallized amylose, and thus the amylose content is a major factor affecting the formation and content of RS₃. However, the amylose content of common starch is relatively low (<30%). Currently, debranching by pullulanase is a common method for increasing the amylose content. Nevertheless, the debranching needs to be performed after starch gelatinization. Due to high viscosity of gelatinized starch, the debranching reaction needs to be performed in a high liquid phase system, the concentration of the starch is generally low (<15 wt %) and the enzymatic hydrolysis time is long. A Chinese patent “Method for Preparing High-Amylose Mung Bean Resistant Starch” (application No. 202111157414.1) discloses a method for preparing mung bean resistant starch using an ultrasonic-enzyme combined method, where the ratio of mung bean starch to water is 1:15 to 1:25 (w/v), and the enzymatic hydrolysis time of the pullulanase is 10.0-14.0 h. A Chinese patent “Method for Preparing Waxy Wheat Resistant Starch” (application No. 202111651682.9) discloses a method for preparing waxy wheat resistant starch using a complex enzyme technology of synergistic action of pullulanase and a branching enzyme, where the mass concentration of starch slurry is 4%-8% and the enzymatic hydrolysis time of the pullulanase is 4-6 h. The lower substrate concentration and longer enzymatic hydrolysis time limit the efficiency of enzyme debranching for improving the amylose content, and cannot meet the requirement of industrialized production of the amylose.

Therefore, it is of important significance to find a new strategy to improve the efficiency of starch debranching to obtain the amylose for the industrialized production of the resistant starch.

### SUMMARY

Aiming at the technical defects in the related art, the present disclosure aims to solve the problem of low yield efficiency of amylose and type III resistant starch (RS₃), and thus provides an extrusion-debranching manner to realize efficient and continuous industrialized production of the amylose and the type III resistant starch. The extrusion is an industrialized continuous thermomechanical production technology with controllable temperature, strong shearing, short time and low cost, and has the advantages of multiple functions and high yield. The present application fully gelatinizes and degrades starch and reduces viscosity thereof through the first extrusion, rapidly and efficiently debranches high-concentration starch through the second extrusion combined with a debranching enzyme so as to obtain amylose, and obtains high-content resistant starch after recrystallization.

The present disclosure aims to provide a method for preparing type III resistant starch in an “extrusion-debranching” manner, including the following steps:
(1) adding a starch raw material into a twin screw extruder for primary extrusion, collecting fully gelatinized starch, and then drying and crushing same to obtain gelatinized starch; and
(2) mixing the gelatinized starch obtained in step (1) with pullulanase, adding the mixture into the twin screw extruder for secondary extrusion, where the water content of the material is set to be 40-60 wt % and the mass concentration of the starch to be 40-60%, performing debranching in the extrusion process, collecting the extruded debranched starch, then recrystallizing same, water washing and centrifuging same, and collecting the precipitate and drying same to obtain type III resistant starch.

In one embodiment, the material refers to all the substances in the twin screw extruder.

In one embodiment, the rotating speed of the twin screw extruder in step (1) is 100-250 rpm, the temperature of 6 temperature regions of the extruder is sequentially raised in the range of 35-120° C., and the water content of the material is set to be 30-60 wt %.

In one embodiment, the starch in step (1) includes one or more of common starch and waxy starch.

In one embodiment, the drying in step (1) can be performed in a conventional drying apparatus at the temperature of 40-50° C., preferably 45° C.

In one embodiment, the rotating speed of the twin screw extruder in step (2) is 100-250 rpm, the temperature of the first 5 temperature regions of the extruder is 35-70° C. and the temperature of the last temperature region is 95-100° C.; preferably, the temperature of the first 5 temperature regions is 35° C., 45° C., 55° C., 60° C. and 65° C., respectively, and the temperature of the last temperature region is 95° C.

In one embodiment, the addition amount of the pullulanase in step (2) is 20-100 ∪/g (based on the dry basis of starch), preferably 80 ∪/g.

In one embodiment, the recrystallization in step (2) is performed at the temperature of 2-25° C. for 10-36 h, preferably at 4° C. for 24 h.

In one embodiment, the centrifugation in step (2) is performed at the centrifugal force of 2,000-5,000 g for 5-20 min.

In one embodiment, the drying in step (2) can be performed in a conventional drying apparatus at the temperature of 40-50° C., preferably 45° C.

Another objective of the present disclosure is to provide type III resistant starch prepared by the above method.

A third objective of the present disclosure is to provide use of the above method in the field of food preparation.

### Beneficial Effects of the Present Disclosure:

(1) The method for efficiently preparing type III resistant starch in an “extrusion-debranching” manner of the present disclosure of the present disclosure uses the synergistic effect of the extrusion technology and pullulanase so as to obtain the high-content amylose and resistant starch through two extrusions, and significantly reduces a digestion performance of the starch. The method is simple and efficient, can rapidly and efficiently debranch high-concentration starch, overcomes the problems in the related art that the content of the substrate starch (less than 15 wt %) cannot be too high and the debranching time is long, and can realize the industrialized continuous production.

(2) The optimum pH value range of the pullulanase is 4.2-4.8. When the starch is debranched, generally hydrochloric acid is added or the starch is dispersed in a buffer salt solution to adjust the pH value. However, in the extrusion system, due to the buffer specificity of the starch matrix, the high-concentration starch enables the pullulanase to have a broader optimum pH range than that in a liquid system. The present disclosure uses high starch concentration extrusion and debranching, reducing the dependence of the pullulanase on pH and simplifying the production process.

(3) The present disclosure will provide theoretical basis and practical guidance for the continuous industrialized production of the amylose, and provides feasible strategies and methods for industrially producing the resistant starch and developing low-glycemic index food.

### DETAILED DESCRIPTION

The technical solution in the examples of the present disclosure will be clearly and completely described below with reference to the examples of the present disclosure. It is obvious that the described examples are only a part of the examples of the present disclosure, and not all of the examples. All other examples obtained by a person of ordinary skill in the art based on the examples of the present disclosure without creative efforts shall fall within the protection range of the present disclosure.

The twin screw extruder involved in the present disclosure is a hot-melting twin screw extruder ZE-16 and purchased from Antos Nanotechnology (Suzhou) Co., Ltd. 6 heating temperature regions are arranged between a feeding end and an extrusion die head of the twin screw extruder, the temperature is sequentially raised from the 1^{st} temperature region to the 6^{th} temperature region, the diameter of a screw is 15.6 mm, and the length-diameter ratio is 25:1.

The extrusion principle of the hot-melting twin screw extruder is as follows: 2 or more than 2 materials are added into a barrel with the temperature-controlled sections, the twin screw is arranged in the barrel, different unit operations are sequentially executed on the twin screw from a feeding part to a machine head, the materials move forward under the propelling of the twin screw and are melted or softened in a certain section, and melts are uniformly mixed under the action of a shearing element and a mixing element, and then extruded from the die head at a certain pressure, speed and shape.

An Englyst in-vitro simulated digestion involved in the present disclosure specifically includes the following processes: 500 mg of a sample is dispersed in 10 mL of a sodium acetate buffer (pH 5.2, 0.2 mol/L) and then gelatinized in a boiling water bath for 30 min while vortexed and stirred at a high speed. The gelatinized sample is equilibrated in a 37° C. water bath for 10 min and then 500 μL of the sample is taken out and added into 5 mL of absolute ethanol to inactivate enzymes (a point is taken at this time as 0 min). Then 5 mL of a mixed enzyme solution (pancreatin and amyloglucosidase) is added into the sample solution, the reaction is performed under a water bath at 37° C. and 170 r/min under oscillation. An enzymatic hydrolysate (500 μL) is collected at 20 min, 40 min, 60 min, 90 min, 120 min, 150 min and 180 min respectively and added into 5 mL of absolute ethanol. Thereafter, the glucose content is analyzed using a glucose oxidase-peroxidase kit. Preparation of mixed enzyme solution: 3 g of the pancreatin is dispersed in 20 mL of distilled water and centrifuged at 3,500 g for 15 min, and 15 mL of a supernatant is collected and uniformly mixed with 1.1 mL of the amyloglucosidase. The content of rapidly digestible starch (RDS), slowly digestible starch (SDS) and resistant starch (RS) is calculated according to the following equations:

$$RDS ( \% ) = ( {G}_{20} - {G}_{0} ) \times 0.9 / TS \times 100 \% \qquad ( 1 )$$

$$SDS ( \% ) = ( {G}_{120} - {G}_{0} ) \times 0.9 / TS \times 100 \% \qquad ( 2 )$$

$$RS ( \% ) = ( TS - RDS - SDS ) \times 0.9 / TS \times 100 \% \qquad ( 3 )$$

where G₀, G₂₀ and G₁₂₀ respectively represent the glucose content in the system at t=0 min, 20 min and 120 min, and TS is the mass of the total starch.

### Example 1

A method for preparing type III resistant starch in an “extrusion-debranching” manner specifically included the following steps:

(1) common corn starch was added into a feeding end of a twin screw extruder for extrusion, where the water content of the material was set to be 40 wt %, the rotating speed of a screw to be 150 rpm, the temperatures of regions I, II, Ill, IV, V and VI of the extruder (ZE-16) respectively to be 35° C., 45° C., 60° C., 80° C., 100° C. and 120° C., and fully gelatinized corn starch was collected, dried at 45° C. and crushed; and

(2) the gelatinized corn starch in step (1) was mixed with 20 ∪/g of pullulanase (calculated by the mass of the dry matter of common corn starch), the mixture was added into the feeding end of the twin screw extruder for extrusion, where the water content of the material was set to be 60 wt %, the mass concentration of the corn starch to be 40%, the temperatures of regions I, II, III, IV, V and VI of the extruder respectively to be 35° C., 45° C., 55° C., 60° C., 65° C. and 95° C., and the rotating speed of the screw to be 150 rpm, the debranched corn starch was collected, recrystallized at 4° C. for 24 h, centrifuged at 4,000 g, and washed with water for 10 min to remove a soluble short chain therein, and the precipitate was collected and dried at 45° C. to obtain resistant starch.

When the pullulanase content was 20 ∪/g, the amylose content in a sample was measured to be 71.53% using a Megazyme amylose and amylopectin assay kit, and the resistant starch content in the sample was measured to be 26.49% using an Englyst in-vitro simulated digestion method. The results were respectively shown in Tables 1 and 2.

### Example 2

A method for preparing type III resistant starch in an “extrusion-debranching” manner specifically included the following steps:
(1) common corn starch was added into a feeding end of a twin screw extruder for extrusion, where the water content of the material was set to be 40 wt %, the rotating speed of a screw to be 150 rpm, the temperatures of regions I, II, Ill, IV, V and VI of the extruder (ZE-16) respectively to be 35° C., 45° C., 60° C., 80° C., 100° C. and 120° C., and fully gelatinized corn starch was collected, dried at 45° C. and crushed; and
(2) the gelatinized corn starch in step (1) was mixed with 40 ∪/g of pullulanase (calculated by the mass of the dry matter of common corn starch), the mixture was added into the feeding end of the twin screw extruder for extrusion, where the water content of the material was set to be 60 wt %, the mass concentration of the corn starch to be 40%, the temperatures of regions I, II, III, IV, V and VI of the extruder respectively to be 35° C., 45° C., 55° C., 60° C., 65° C. and 95° C., and the rotating speed of the screw to be 150 rpm, the debranched corn starch was collected, recrystallized at 4° C. for 24 h, centrifuged at 4,000 g, and washed with water for 10 min to remove a soluble short chain therein, and the precipitate was collected and dried at 45° C. to obtain resistant starch.

When the pullulanase content was 40 ∪/g, the amylose content in a sample was measured to be 75.34% using a Megazyme amylose and amylopectin assay kit, and the resistant starch content in the sample was measured to be 29.67% using an Englyst in-vitro simulated digestion method. The results were respectively shown in Tables 1 and 2.

### Example 3

A method for preparing type III resistant starch in an “extrusion-debranching” manner specifically included the following steps:
(1) common corn starch was added into a feeding end of a twin screw extruder for extrusion, where the water content of the material was set to be 40 wt %, the rotating speed of a screw to be 150 rpm, the temperatures of regions I, II, Ill, IV, V and VI of the extruder (ZE-16) respectively to be 35° C., 45° C., 60° C., 80° C., 100° C. and 120° C., and fully gelatinized corn starch was collected, dried at 45° C. and crushed; and
(2) the gelatinized corn starch in step (1) was mixed with 60 ∪/g of pullulanase (calculated by the mass of the dry matter of common corn starch), the mixture was added into the feeding end of the twin screw extruder for extrusion, where the water content of the material was set to be 60 wt %, the mass concentration of the corn starch to be 40%, the temperatures of regions I, II, III, IV, V and VI of the extruder respectively to be 35° C., 45° C., 55° C., 60° C., 65° C. and 95° C., and the rotating speed of the screw to be 150 rpm, the debranched corn starch was collected, recrystallized at 4° C. for 24 h, centrifuged at 4,000 g, and washed with water for 10 min to remove a soluble short chain therein, and the precipitate was collected and dried at 45° C. to obtain resistant starch.

When the pullulanase content was 60 ∪/g, the amylose content in a sample was measured to be 80.70% using a Megazyme amylose and amylopectin assay kit, and the resistant starch content in the sample was measured to be 35.31% using an Englyst in-vitro simulated digestion method. The results were respectively shown in Tables 1 and 2.

### Example 4

A method for preparing type III resistant starch in an “extrusion-debranching” manner specifically included the following steps:
(1) common corn starch was added into a feeding end of a twin screw extruder for extrusion, where the water content of the material was set to be 40 wt %, the rotating speed of a screw to be 150 rpm, the temperatures of regions I, II, Ill, IV, V and VI of the extruder (ZE-16) respectively to be 35° C., 45° C., 60° C., 80° C., 100° C. and 120° C., and fully gelatinized corn starch was collected, dried at 45° C. and crushed; and
(2) the gelatinized corn starch in step (1) was mixed with 80 ∪/g of pullulanase (calculated by the mass of the dry matter of common corn starch), the mixture was added into the feeding end of the twin screw extruder for extrusion, where the water content of the material was set to be 60 wt %, the mass concentration of the corn starch to be 40%, the temperatures of regions I, II, III, IV, V and VI of the extruder respectively to be 35° C., 45° C., 55° C., 60° C., 65° C. and 95° C., and the rotating speed of the screw to be 150 rpm, the debranched corn starch was collected, recrystallized at 4° C. for 24 h, centrifuged at 4,000 g, and washed with water for 10 min to remove a soluble short chain therein, and the precipitate was collected and dried at 45° C. to obtain resistant starch.

When the pullulanase content was 80 ∪/g, the amylose content in a sample was measured to be 85.27% using a Megazyme amylose and amylopectin assay kit, and the resistant starch content in the sample was measured to be 40.39% using an Englyst in-vitro simulated digestion method. The results were respectively shown in Tables 1 and 2.

### Example 5

A method for preparing type III resistant starch in an “extrusion-debranching” manner specifically included the following steps:
(1) common corn starch was added into a feeding end of a twin screw extruder for extrusion, where the water content of the material was set to be 40 wt %, the rotating speed of a screw to be 150 rpm, the temperatures of regions I, II, Ill, IV, V and VI of the extruder (ZE-16) respectively to be 35° C., 45° C., 60° C., 80° C., 100° C. and 120° C., and fully gelatinized corn starch was collected, dried at 45° C. and crushed; and
(2) the gelatinized corn starch in step (1) was mixed with 100 ∪/g of pullulanase (calculated by the mass of the dry matter of common corn starch), the mixture was added into the feeding end of the twin screw extruder for extrusion, where the water content of the material was set to be 60 wt %, the mass concentration of the corn starch to be 40%, the temperatures of regions I, II, III, IV, V and VI of the extruder respectively to be 35° C., 45° C., 55° C., 60° C., 65° C. and 95° C., and the rotating speed of the screw to be 150 rpm, the debranched corn starch was collected, recrystallized at 4° C. for 24 h, centrifuged at 4,000 g, and washed with water for 10 min to remove a soluble short chain therein, and the precipitate was collected and dried at 45° C. to obtain resistant starch.

When the pullulanase content was 100 ∪/g, the amylose content in a sample was measured to be 90.21% using a Megazyme amylose and amylopectin assay kit, and the resistant starch content in the sample was measured to be 41.50% using an Englyst in-vitro simulated digestion method. The results were respectively shown in Tables 1 and 2.

### Example 6

A method for preparing type III resistant starch in an “extrusion-debranching” manner specifically included the following steps:
(1) common corn starch was added into a feeding end of a twin screw extruder for extrusion, where the water content of the material was set to be 40 wt %, the rotating speed of a screw to be 150 rpm, the temperatures of regions I, II, Ill, IV, V and VI of the extruder (ZE-16) respectively to be 35° C., 45° C., 60° C., 80° C., 100° C. and 120° C., and fully gelatinized corn starch was collected, dried at 45° C. and crushed; and
(2) the gelatinized corn starch in step (1) was mixed with 80 ∪/g of pullulanase (calculated by the mass of the dry matter of common corn starch), the mixture was added into the feeding end of the twin screw extruder for extrusion, where the water content of the material was adjusted and set to be 40 wt %, the mass concentration of the corn starch to be 60%, the temperatures of regions I, II, III, IV, V and VI of the extruder respectively to be 35° C., 45° C., 55° C., 60° C., 65° C. and 95° C., and the rotating speed of the screw to be 150 rpm, the debranched corn starch was collected, recrystallized at 4° C. for 24 h, centrifuged at 4,000 g, and washed with water for 10 min to remove a soluble short chain therein, and the precipitate was collected and dried at 45° C. to obtain resistant starch.

When the mass concentration of the corn starch was 60%, the amylose content in a sample was measured to be 74.94% using a Megazyme amylose and amylopectin assay kit, and the resistant starch content in the sample was measured to be 29.78% using an Englyst in-vitro simulated digestion method. The results were respectively shown in Tables 1 and 2.

### Comparative Example 1

Common corn starch was added into a feeding end of a twin screw extruder for extrusion, where the water content was set to be 40 wt %, the rotating speed of a screw to be 150 rpm, the temperatures of regions I, II, III, IV, V and VI of the extruder (ZE-16) respectively to be 35° C., 45° C., 60° C., 80° C., 100° C. and 120° C., and fully gelatinized corn starch was collected, dried at 45° C. and crushed to obtain the finished product.

The amylose content in comparative example 1 was measured to be 29.52% using a Megazyme amylose and amylopectin assay kit, and the resistant starch content in the sample was measured to be 5.61% using an Englyst in-vitro simulated digestion method. The results were respectively shown in Tables 1 and 2.

### Comparative Example 2

(1) common corn starch was added into a feeding end of a twin screw extruder for extrusion, where the water content was set to be 40 wt %, the rotating speed of a screw to be 150 rpm, the temperatures of regions I, II, III, IV, V and VI of the extruder (ZE-16) respectively to be 35° C., 45° C., 60° C., 80° C., 100° C. and 120° C., and fully gelatinized corn starch was collected, dried at 45° C. and crushed; and
(2) the gelatinized corn starch was added into the feeding end of the twin screw extruder for extrusion, where the water content was set to be 60 wt %, the temperatures of regions I, II, III, IV, V and VI of the extruder respectively to be 35° C., 45° C., 55° C., 60° C., 65° C. and 95° C., the rotating speed of a screw to be 150 rpm, and the extrudate was collected, recrystallized at 4° C. for 24 h and dried at 45° C. to obtain the finished product.

### Comparative Example 3

(1) common corn starch was added into a feeding end of a twin screw extruder for extrusion, where the water content of the material was set to be 40 wt %, the rotating speed of a screw to be 150 rpm, the temperatures of regions I, II, III, IV, V and VI of the extruder (ZE-16) respectively to be 35° C., 45° C., 60° C., 80° C., 100° C. and 120° C., and fully gelatinized corn starch was collected, dried at 45° C. and crushed; and
(2) the dried and crushed corn starch in step (1) was mixed with 20 ∪/g of pullulanase (calculated by the mass of the dry matter of common corn starch) and water was added to ensure that the mass concentration of the corn starch was 40%; and at this time, the system was in a viscous solid state and the enzymolysis reaction under stirring cannot be performed.

The amylose content in comparative example 2 was measured to be 31.61% using a Megazyme amylose and amylopectin assay kit, and the resistant starch content in the sample was measured to be 8.16% using an Englyst in-vitro simulated digestion method. The results were respectively shown in Tables 1 and 2.

TABLE 1
Amylose content of samples

| Sample | Amylose content (%) |
|---|---|
| Corn starch | 25.80 ± 2.40^{f} |
| Comparative example 1 | 29.52 ± 1.61^{ef} |
| Comparative example 2 | 31.61 ± 2.72^{e} |
| Example 1 | 71.53 ± 1.84^{d} |
| Example 2 | 75.34 ± 1.24^{cd} |
| Example 3 | 80.70 ± 3.73^{bc} |
| Example 4 | 85.27 ± 1.25^{ab} |
| Example 5 | 90.21 ± 1.37^{a} |
| Example 6 | 74.94 ± 1.97^{d} |

(Note: Different letters, ^{a}, ^{b}, ^{c}, ^{d}, ^{e} and ^{f}, indicated significant differences between each group of data, p < 0.05)

TABLE 2
Content of rapidly digestible starch, slowly digestible
starch and resistant starch in samples

| | Rapidly | Slowly | |
|---|---|---|---|
| | digestible | digestible | Resistant |
| Sample | starch (%) | starch (%) | starch (%) |
| Corn starch | 89.16 ± 2.08^{a} | 7.45 ± 1.49^{e} | 3.38 ± 0.58^{f} |
| Comparative | 85.00 ± 2.02^{a} | 9.38 ± 1.16^{e} | 5.61 ± 0.86^{ef} |
| example 1 | | | |
| Comparative | 76.45 ± 2.67^{b} | 15.39 ± 1.18^{d} | 8.16 ± 1.48^{e} |
| example 2 | | | |
| Example 1 | 54.94 ± 2.65^{c} | 18.56 ± 1.88^{cd} | 26.49 ± 0.77^{d} |
| Example 2 | 49.38 ± 0.58^{d} | 20.94 ± 1.32^{bc} | 29.67 ± 0.73^{c} |
| Example 3 | 43.71 ± 1.26^{e} | 20.97 ± 0.50^{bc} | 35.31 ± 1.76^{b} |
| Example 4 | 36.67 ± 2.19^{f} | 22.94 ± 0.55^{ab} | 40.39 ± 1.64^{a} |
| Example 5 | 33.89 ± 1.88^{f} | 24.61 ± 2.60^{a} | 41.50 ± 0.72^{a} |
| Example 6 | 48.39 ± 1.65^{d} | 21.83 ± 0.70^{ab} | 29.78 ± 2.36^{c} |

(Note: Different letters, ^{a}, ^{b}, ^{c}, ^{d}, ^{e} and ^{f}, indicated significant differences between each group of data, p < 0.05)

The method for preparing type III resistant starch of the present disclosure efficiently debranches starch under high starch substrate concentration and short time using extrusion-debranching. A feasible method for industrial production of amylose and resistant starch is provided. After the extrusion-debranching, the amylose content in the starch can be up to 90.21% and the resistant starch content can be up to 41.50%. The method is simple to operate, high in production efficiency and capable of being used in the industrialized continuous production.

The above examples are presented to facilitate a person of ordinary skill in the art to understand and use the present disclosure. Obviously, a person skilled in the art can easily make various modifications to these examples, and apply a general principle described herein to other examples without creative efforts. Therefore, the present disclosure is not limited to the above examples. All improvements and modifications made by a person skilled in the art according to implication of the present disclosure without departing from the scope of the present disclosure should fall within the protection range of the present disclosure.

What is claimed is:

1. A method for preparing type III resistant starch in an “extrusion-debranching” manner, comprising the following steps:
(1) adding a starch raw material into a twin screw extruder for primary extrusion, collecting fully gelatinized starch, and then drying and crushing same to obtain gelatinized starch; and
(2) fully mixing the gelatinized starch obtained in step (1) with pullulanase, adding the mixture into the twin screw extruder for secondary extrusion, wherein the water content of the material is set to be 40-60 wt % and the mass concentration of the starch is set to be 40-60%, performing debranching in the extrusion process, collecting the obtained debranched starch, then recrystallizing same, water washing and centrifuging same, and collecting the precipitate and drying same to obtain type III resistant starch.

2. The method for preparing type III resistant starch in an “extrusion-debranching” manner according to claim 1, wherein a rotating speed of the twin screw extruder in step (1) is 100-250 rpm, the temperature of 6 temperature regions of the twin screw extruder is sequentially raised in the range of 35-120° C., and the water content of the material is set to be 30-60 wt %.

3. The method for preparing type III resistant starch in an “extrusion-debranching” manner according to claim 1, wherein the starch in step (1) comprises one or more of common starch and waxy starch.

4. The method for preparing type III resistant starch in an “extrusion-debranching” manner according to claim 1, wherein the drying in step (1) is performed in a conventional drying apparatus at the temperature of 40-50° C.

5. The method for preparing type III resistant starch in an “extrusion-debranching” manner according to claim 1, wherein the temperature of the first 5 temperature regions of the twin screw extruder in step (2) is 35-70° C., and the temperature of the last temperature region is 95-100° C.

6. The method for preparing type III resistant starch in an “extrusion-debranching” manner according to claim 1, wherein an addition amount of the pullulanase in step (2) is 20-100 ∪/g based on the dry basis of starch.

7. The method for preparing type III resistant starch in an “extrusion-debranching” manner according to claim 1, wherein the recrystallization in step (2) is performed at the temperature of 2-25° C. for 10-36 hours.

8. The method for preparing type III resistant starch in an “extrusion-debranching” manner according to claim 1, wherein the centrifugation in step (2) is performed at the centrifugal force of 2,000-5,000 g for 5-20 minutes.

9. The method for preparing type III resistant starch in an “extrusion-debranching” manner according to claim 1, wherein the drying in step (2) is performed in a conventional drying apparatus at the temperature of 40-50° C.
